(19) United States
(12) Patent Application Publication
(10) Pub. No.: US 2013/0316014 A1
(43) Pub. Date: Nov. 28, 2013

(54) METHODS FOR PREDICTING PROGNOSIS OF A SUBJECT WITH A MYELOID MALIGNANCY

(71) Applicants: Jaroslaw P. Maciejewski, Cleveland, OH (US); Hideki Makishima, South Euclid, OH (US); Ramon Tiu, Cleveland, OH (US); Valeria Visconte, Cleveland, OH (US)

(72) Inventors: Jaroslaw P. Maciejewski, Cleveland, OH (US); Hideki Makishima, South Euclid, OH (US); Ramon Tiu, Cleveland, OH (US); Valeria Visconte, Cleveland, OH (US)

(21) Appl. No.: 13/855,217

(22) Filed: Apr. 2, 2013

Related U.S. Application Data
(60) Provisional application No. 61/619,249, filed on Apr. 2, 2012.

(51) Int. Cl. C12Q 1/68

(52) U.S. Cl. CPC C12Q 1/6886
USPC 424/577; 506/2; 436/501

(57) ABSTRACT

One aspect of the present disclosure includes a method for predicting the prognosis of a subject with a myeloid malignancy. One step of the method includes obtaining a biological sample from the subject. Next, at least one mutation in a spliceosome-associated protein, or a polynucleotide encoding the spliceosome-associated protein that results in defective splicing can be detected in the biological sample. The presence of at least one mutation in the spliceosome-associated protein, or a polynucleotide encoding the spliceosome-associated protein, is indicative of the subject's prognosis.

### RELATED APPLICATION

This application claims the benefit of U.S. Provisional Patent Application Ser. No. 61/619,249, filed Apr. 2, 2012, the entirety of which is hereby incorporated by reference in its entirety for all purposes.

### TECHNICAL FIELD

The present disclosure relates generally to methods for predicting the prognosis of a subject with a myeloid malignancy, and more particularly to a method for predicting the prognosis of a subject with a myelodysplastic syndrome or leukemia based on certain predicative parameters, such as mutations in the spliceosomal machinery.

### BACKGROUND

The myelodysplastic syndromes (MDS) are characterized by clonal hematopoiesis, a variety of chromosomal abnormalities, bone marrow failure and a propensity for evolution to acute myeloid leukemia. Because of their often protracted course, MDS recapitulate the stages of acquisition of a malignant phenotype, thereby offering insights into leukemogenesis. While traditionally, histomorphology-based schemes have been applied to sub-classify MDS patients, this approach is unlikely to be reflective of the underlying pathogenesis.

Instead, a better molecular characterization of MDS on the genomic, epigenetic and genetic levels is more likely to objectively diagnose patients, determine their prognosis and, based on the underlying molecular defects, direct the application of targeted therapies. The emerging realization of the molecular diversity of MDS parallels the clinical and phenotypic heterogeneity of this disease. Moreover, molecular defects have the potential to serve as biomarkers and are more likely to be suitable for the identification of therapy targets and responsiveness/refractoriness to treatment.

The application of high-throughput molecular technologies, including high density single nucleotide polymorphism arrays (SNP-A) and new sequencing technologies has led to improved characterization of genomic lesions, such as chromosomal aberrations and somatic mutations affecting specific classes of genes, including signal transducers (e.g., CBL), apoptotic genes (e.g., TP53 and RAS), genes involved in epigenetic regulation of DNA (e.g., DNMT3A, IDH1/2 and TET2) and histone modifiers (e.g., EZH2, UTX and ASXL1). While some mutations in these factors are activating, most are loss of function or hypomorphic mutations and affect bona fide tumor suppressor genes (TSG). Of greatest diagnostic impact are recurrent mutations found in specific genes. Most TSG mutations are not canonical, though, making systematic clinical diagnostics more difficult.

### SUMMARY

One aspect of the present disclosure includes a method for predicting the prognosis of a subject with a myeloid malignancy. One step of the method includes obtaining a biological sample from the subject. Next, at least one mutation in a spliceosome-associated protein, or a polynucleotide encoding the spliceosome-associated protein that results in defective splicing can be detected in the biological sample. The presence of at least one mutation in the spliceosome-associated protein, or a polynucleotide encoding the spliceosome-associated protein, is indicative of the subject's prognosis.

Another aspect of the present disclosure includes a method for diagnosing a subject with a high risk MDS or leukemia. One step of the method includes obtaining a biological sample from the subject. Next, the presence of at least one mutation in a SRSF2 protein, or a polynucleotide encoding the SRSF2 protein that results in defective splicing can be detected in the biological sample. The presence of at least one mutation in the SRSF2 protein, or a polynucleotide encoding the SRSF2 protein, is indicative of a high or higher-risk MDS in the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1A-E show somatic spliceosomal gene (U2AF1, SF3B1, SRSF2, LUC7L2, PRPF8 and ZRSR2) mutations as detected by next-generation sequencing (NGS) and Sanger sequencing technologies. Using a NGS-based whole exome sequencing analysis of whole bone marrow DNA from a patient with RCMD (left), a mutation of U2AF1 (21q22.3) at position 44,514,777 (T>C) was detected in 13 of 18 reads. Analysis of DNA from CD3 positive cells showed a much lower frequency of the base change (2 out of 15 reads, right), highlighting the somatic nature of this alteration. The finding was confirmed by Sanger sequencing. Arrows and bars indicate the specific nucleotide and predicted codon, respectively. It should be noted that U2AF1 is expressed from the minus strand, and therefore the NGS sequencing presentation (upper panels) is complementally reversed in Sanger sequencing results (middle panels). This heterozygous somatic mutation results in the predicted nucleotide change 470 A>G in exon 6 of the coding region, which lead to the amino acid change Q157R in the second zinc finger domain. In the entire cohort, 27 mutations were observed in 26 patients, including a whole gene deletion. All 26 missense mutations were located in one of the 2 zinc finger domains (ZNF); 2 residues, S34 or Q157, were frequently affected (lower figures). The conceptual structure of 3 U2AF1 isoforms, including the 2 ZNFs and the RNA recognition motif (RRM), and mutation locations are indicated (FIG. 1A). Using a NGS-based whole exome sequencing analysis of bone marrow DNA from a patient with CMML (middle left), a mutation of SF3B1 (2q33.1) at position 198,267,491(C>G) was detected in 9 out of 24 reads. The somatic nature of this alteration was confirmed by an analogous analysis of the CD3+ (T-cell rich) fraction, with the change being less frequent (2 out of 23 reads) (middle right). The mutation was confirmed by conventional Sanger sequencing (bottom). Arrows and bars indicate the specific nucleotide and predicted codon, respectively. This heterozygous somatic mutation results in the nucleotide change 1866 G>T in exon 14 of SF3B1, resulting in the amino acid change E622D in the HSH155 domain. SF3B1 is expressed from the minus strand, so the NGS sequencing results (middle panels) are complementally reversed in comparison to the Sanger sequencing (bottom panels). Analysis of the entire cohort identified mutations in 33 patients, including a case with a whole gene deletion. All 31 missense and 1 deletion mutations were located in the HSH155 domain, which is necessary for U2 snRNP function in prespliceosome assembly; 3 residues (E622, K666 or K700) were affected frequently (top) (FIG. 1B). Further screening by NGS led to the detection of a nonsense mutation (R27X) in LUC7L2 (ch7q34) (top), which participates in the recognition of splice donor sites in association with the U1 snRNP spliceosomal subunit, and a missense mutation (M13071) in PRPF8 (ch17p13.3) (bottom), which is a large U5 snRNP-specific protein essential for pre-mRNA splicing. Whole genome sequence results were confirmed by Sanger sequencing. RS, U5 2-snRNA bdg and MPN indicate serine/arginine-rich domain, U5-snRNA binding site 2, and Mpr1p, Fadi p N-terminal domain, respectively (FIG. 10). Mutations of SRSF2, an arginine/serine-rich splicing factor, were detected in 29 cases among the entire cohort, including 2 whole gene deletions and a microdeletion within the gene (top). All mutations were heterozygous and affected P95, which is close to the RNA recognition motif (RRM). The somatic nature of the P95R mutation was confirmed using whole bone marrow and T-cell rich fraction DNAs (bottom) (FIG. 1D). A nonsense mutation (W153X) was found in ZRSR2, another arginine/serine-rich splicing regulatory factor, in a case of CMML. ZRSR2 is located at chXp22.2 and the nonsense mutation was hemizygous in this male case (whole bone marrow) (FIG. 1E);

FIG. 2 shows frequency and phenotypic association of spliceosomal mutations in myeloid malignancies. In the entire cohort (N=310), a total of 88 spliceosome pathway mutations (U2AF1, SF3B1, SRSF2) were observed in every subtype of myeloid malignancies, except for MPN. In low risk MDS, SF3B1 mutations were most frequent among the 3 genes. In particular, SF3B1 was mutated in 15/20 cases of RARS (60%). In the high risk MDS and AML group, U2AF1 mutations were most frequent (15/139; 10.8%). In the MDS/MPN group, SRSF2 was most frequently mutated (13/46; 28.2%), while SF3B1 is mutated at a high frequency in RARS-t (10/11; 90.1%);

FIGS. 3A-C show the impact of spliceosomal mutations on clinical outcomes. In the entire cohort, patients with U2AF1 mutations (MT) had worse overall survival (OS), compared with wild type (WT), but SF3B1 mutations made OS significantly shorter (FIG. 3A). In low risk MDS, mutation of SF3B1 was a good prognostic factor but SRSF2 mutations are associated with worse prognosis (FIG. 3B). In MDS/MPN, patients with mutated U2AF1 had a shorter OS, but SF3B1 mutations were associated with significantly better prognosis. In addition, SRSF2 mutations did not affect outcomes (FIG. 3C);

FIGS. 4A-C show unsplicing of specific genes due to spliceosomal mutations as detected by deep RNA sequencing. Next generation-based-RNA deep sequencing was used to quantitatively study the splicing pattern of all transcripts from a number of genes (FIG. 4A). The upper panel shows the intron 5 and exon 6 boundary of TET2 (dotted line). Green and blue reads represent the 3′ to 5′ and 5′ to 3′ directions, respectively. 5 reads correspond to transcripts which were not spliced (unspliced; black circle) and 4 were spliced (white circle) at this boundary. The lower panel shows read counts at the 5′ and 3′ splice sites of each intron (3-10) of TET2. White and black bars indicate the number of spliced and unspliced reads, respectively. In a case of AML with a U2AF1 mutation, more unspliced than spliced reads were observed at the 3′ splice site of intron 5 (left panel), likely due to a loss of spliceosome function. However, unspliced RNAs were less frequent than spliced RNAs in WT RNA sequencing (right panel) (FIG. 4B). At both the 3′ and 5′ splice sites of RUNX1 intron 6, unspliced reads were more frequent than spliced reads in an AML case with a U2AF1 mutation (left). However there were fewer unspliced transcripts at the same site in a U2AF1 WT sample (right). In TP53, all of the introns showed a normal splicing pattern in both U2AF1 mutant (left) and WT (right) samples (FIG. 4C);

FIGS. 5A-F show various members of spliceosomal machinery can be affected by somatic mutations in myeloid malignances. Initially, the U2 auxiliary factor (U2AF) complex recognizes the branch point site. In detail, the smaller subunit of U2AF (U2AF1) binds to the 3′ AG dinucleotide of the intron (splice acceptor site), while the larger subunit, U2AF2, binds to the polypyrimidine sequence ((C/U)n). SF1 binds to the branch point sequence including the branch ‘A’ nucleotide in the upstream of (C/U)n. ZRSR2 and U2AF26 also interact with U2AF to perform essential functions in U2 RNA splicing. Arginine/serine-rich splicing factors SRSF2 and SRSF6 bind to polypurine sequences ((A/G)n) in the exon. SRSF2 interacts with U2AF1. SON, a recently discovered spliceosomal gene, mediates constitutive splicing of weak splice sites (FIG. 5A). U2AF2, SF1, ZRSR2 and U2AF26 leave the site, while the U2 snRNP, along with SF3A1, SF3B1 and SAP130, bind to the 3′ intron boundary. LUC7L2 is associated with the U1 snRNP complex which recognizes the GU dinucleotide at the 5′ splice donor site. PRPF8 plays an essential role in the interaction among U4/U6/U5 snRNPs, while HCFC1 contributes to the U1/U5 interaction (FIG. 5B). The U1, U5, U4/U6 and U2 snRNPs are assembled to form the spliceosome. The intron is bent and folded to bring the splice donor site and branch point close together (FIG. 5C). After the U1 and U4 snRNPs detach and leave the spliceosome, the branch point nucleotide within the intron defined during spliceosome assembly performs a nucleophilic attack on the first nucleotide of the intron at the 5′ splice donor site, forming the lariat intermediate (FIG. 5D). The hydroxyl (OH) of the released 5′ exon then performs a nucleophilic attack at the last nucleotide of the intron at the 3′ splice acceptor site, thus joining the exons and releasing the intron lariat (FIG. 5E). The mutated components in myelodysplasia are indicated by stars in FIGS. 5A-E and prevalence of mutations and references were presented in the table (FIG. 5F);

FIG. 6 shows a U2AF1 mutation in a patient with trisomy 21. In a patient with secondary AML with trisomy 21 (upper panel), the mutation (c; 470 A>G, Q157R) was detected in 2 out of 3 alleles of U2AF1 using whole bone marrow with 85% blasts (bottom left). After chemotherapy, the blasts decreased in the bone marrow (10%) and the mutation was less prevalent (lower right);

FIG. 7 shows the somatic nature of a U2AF1 mutation of the first zinc finger domain. A S34F heterozygous mutation was seen in the whole bone marrow of a patient with sAML, but that mutated clone was less common in the CD3− rich fraction (lower left);

FIG. 8 shows the impact of spliceosomal mutations on clinical outcomes in acute myeloid leukemia;

FIG. 9 shows ancestral acquired nature of an U2AF1 mutation in the course of evolution to secondary AML. In another patient, a Q157P heterozygous mutation was initially found at the sAML stage (blast 79%) and was also detected in the primary low risk MDS (RCMD) phase (blast 2%) (lower right);

FIG. 10 shows the results of RNA NGS in patients with U2AF1 mutations as compared to WT controls. Next generation-based-RNA deep sequencing quantitatively showed splicing pattern of transcript in whole human gene. The upper panels show the results of U2AF1 mutant case. Dotted lies indicate intron 5 and exon 6 boundary of TET2 in two pannels. Green and blue reads represent the 3′ to 5′ and 5′ to 3′ directions, respectively. 9 reads correspond to transcripts which were not spliced (unspliced; black circle) and 6 were spliced (white circle) at this boundary in the right (the zoomed-in-figure). The lower panels show U2AF1 wild type case, presenting less reads with unspliced mRNA (black circle);

FIG. 11 shows evaluation of RNA splicing in a patient with a U2AF26 mutation. At both the 3′ and 5′ splice sites of RUNX1 intron 4, 5, 6, unspliced reads were more frequent than spliced reads in an AML case with a U2AF26 mutation;

FIG. 12 shows the results of alternative splicing analysis with mRNA NGS in patients with U2AF1 mutations as compared to WT controls. Next generation-based-RNA deep sequencing showed alternative splicing pattern of the exon 9 of FECH gene. This exon was skipped in U2AF1 mutant cases (MT), but not in wild type cases (WT); and

FIGS. 13A-D show theoretical splicing abnormalities; unsplicing and exon skipping. Normal splicing removes introns to join the three exons without any retention of intronic sequences (FIG. 13A). If pre-mRNA splicing is not successful at the normal 3′ splice site of intron 1, 3′ splicing will occur at a new splice site leading to the retention of an unspliced portion of the 3′ intron between exon 1 and 2 (FIG. 13B). Unsplicing of the 5′ end of intron 1 results in the retention of a 5′ intronic sequences between exon 1 and 2 (FIG. 13C). Exon skipping cuts off two introns and an exon between the introns altogether and creates a mRNA composed of two exons instead of 3 (FIG. 13D).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present disclosure pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the present disclosure.

In the context of the present invention, the term “protein” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms

As used herein, the term “spliceosome” can refer to a ribonucleoprotein complex that removes introns from one or more pre-mRNA segments. Mammalian spliceosomes are complex structures, containing over 150 distinct proteins and 5 small nuclear RNAs. Thus, the term “spliceosome-associated protein” as used herein can refer to any polypeptide or protein comprising a spliceosome.

As used herein, the term “subject” can refer to any animal, including, but not limited to, humans and non-human animals (e.g., rodents, arthropods, insects, fish), non-human primates, ovines, bovines, ruminants, lagomorphs, porcines, caprines, equines, canines, felines and ayes.

As used herein, the terms “polynucleotide” or “polynucleotides” can refer to a gene, oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material natural or synthetic in origin, including, e.g., iRNA, siRNA, microRNA, ribonucleoproteins (e.g., iRNPs). The term can also encompass nucleic acids, i.e., oligonucleotides, containing known analogues of natural nucleotides. Additionally, the term can encompass nucleic acid-like structures with synthetic backbones.

As used herein, the terms “detection” or “detecting” are used in the broadest sense and can include both qualitative and quantitative measurements of a spliceosomal-associated protein or a polynucleotide encoding a spliceosomal-associated protein.

As used herein, the term “biological sample” is used herein in its broadest sense and can refer to a bodily sample obtained from a subject (e.g., a human) or from components (e.g., tissues) of a subject. The biological sample may be of any biological tissue or fluid with which at least one mutation in a spliceosomal-associated protein, or a polynucleotide encoding a spliceosomal-associated protein, may be assayed. For example, the biological sample can include a “clinical sample”, i.e., a sample derived from a subject. Such samples can include, but are not limited to: peripheral bodily fluids, which may or may not contain cells, e.g., blood, urine, plasma, mucous, bile pancreatic juice, supernatant fluid and serum; tissue or fine needle biopsy samples; and archival samples with known diagnosis, treatment and/or outcome history. Biological samples may also include sections of tissues, such as frozen sections taken from histological purposes. The term “biological sample” can also encompass any material derived by processing the sample. Derived materials can include, but are not limited to, cells (or their progeny) isolated from the biological sample and proteins or polynucleotides extracted from the sample. Processing of the biological sample may involve one or more of, filtration, distillation, extraction, concentration, fixation, inactivation of interfering components, addition of reagents, and the like.

As used herein, the term “myeloid malignancy” can refer to a variety of clonal disorders that are characterized by acquired somatic mutation(s) in hematopoietic progenitor cells, such as myelodysplastic disorders (MDS) and myeloproliferative neoplasms.

As used herein, the terms “myelodysplastic syndrome” or “MDS” can refer to a heterogeneous group of closely related clonal hematopoietic disorders. All are characterized by a hypercellular or hypocellular marrow with impaired morphology and maturation (dysmyelopoiesis) and peripheral blood cytopenias, resulting from ineffective blood cell production. All three cell lineages in myeloid hematopoiesis can be involved, including erythrocytic, granulocytic, and megakaryocytic cell lines.

The present disclosure relates generally to methods for predicting the prognosis of a subject with a myeloid malignancy, and more particularly to a method for predicting the prognosis of a subject with a myelodysplastic syndrome MDS or leukemia based on certain predicative parameters, such as mutations in the spliceosomal machinery. During transcription in eukaryotic cells, pre-mRNA, which contains both intronic and exonic sequences, undergoes removal of introns and ligation of exons, a fundamental process required to form mature mRNA transcripts. Since most human genes contain more than one intron, various intron combinations can be spliced out, a process referred to as alternative splicing. Spliceosomes are intracellular protein-RNA complexes that catalyze all necessary reactions during splicing. During the splicing process, formation of the spliceosome active site involves an ordered, stepwise assembly of discrete particles on the pre-mRNA substrate and the recognition of specific sites (3′ and 5′) in the pre-mRNA.

The present disclosure relates, at least in part, to the discovery of somatic mutations affecting, in a recurrent fashion, genes of the spliceosome machinery that result in defective splicing. Specifically, it was discovered that: (1) SF3B1 mutations are prevalent in low-risk MDS with ring sideroblasts, such as refractory anemia with ring sideroblasts (RARS) and RARS associated with marked thrombocytosis (RARS-T) and helpful in distinguishing clonal causes of RS from non-clonal causes, such as alcohol intake, drug-induced, congenital causes of sideroblastic anemia, and others; (2) SF3B1 mutations are associated with a favorable prognosis in patients with low-risk MDS; (3) U2AF1 mutations are frequent in advanced forms of MDS, such as secondary acute myeloid leukemia (sAML) and chronic myelomonocytic leukemia (CMML); (4) U2AF1 mutations are predicative of shorter survival in patients with CMML; (5) SRSF2 mutations are frequent in myelomonocytic leukemia (e.g., CMML) and advanced forms of MDS, such as sAML and refractory anemia with excess blasts (RAEB); and (6) SRSF2 mutations are associated with worse survival in low-risk MDS. As described in more detail below, and without being bound by any particular theory, in certain aspects of the present disclosure it is believed that the discovery of recurrent somatic mutations in various genes encoding spliceosomal proteins can be used as predicative parameters to assess the prognosis of subjects suffering from certain myeloid malignancies, such as MDS or leukemia.

One aspect of the present disclosure includes a method for predicting the prognosis of a subject with a myeloid malignancy, such as a MDS or leukemia. MDS are bone marrow stem cell disorders resulting in disorderly and ineffective hematopoiesis (blood production) manifested by irreversible quantitative and qualitative defects in hematopoietic (blood-forming) cells. The syndromes may arise de novo, or following treatment with chemotherapy and/or radiation therapy. A MDS or leukemia from which the subject is suffering can generally include any hematological disorder characterized by ineffective production of blood cells and varying risks of transformation to AML.

Classification systems of MDS include the French-American-British (FAB) classification system, the International Prognostic Scoring System (IPSS) that was generated during an International MDS Risk Analysis Workshop (see Greenberg et al., Blood 89:2079-2088, 1997), and the World Health Organization (WHO) classification system, which relies on the appearance of particular cells in the bone marrow. The IPSS, for example, takes into account the number of cytopenias, bone marrow blast percentage, and refined cytogenetic characterization. Each of these three indicators is rated according to its severity and the ratings are combined into a “score”. Scores are then sorted into one of four risk categories: low (0 points); intermediate-1 (0.5 to 1.0 points); intermediate-2 (1.5 to 2.0 points); and high (2.5 to 3.5 points). The two lower categories can be further described as the lower risk group, while the two upper categories can be further described as the higher risk group.

The WHO classification system, like the FAB system, distinguishes the different forms of MDS based on bone marrow and peripheral smear findings as follows:
(1) Refractory anemia (RA)—peripheral smear: anemia, <1% blasts; bone marrow: unilineage erythroid dysplasia (in ≧10% of cells), <5% blasts.
(2) Refractory neutropenia (RN)—<1% of all MDS; peripheral smear: neutropenia, <1% blasts; bone marrow: unilineage granulocytic dysplasia, <5% blasts.
(3) Refractory thrombocytopenia (RT)—<1% of all MDS; peripheral smear: thrombocytopenia, <1% blasts; bone marrow: unilineage megakaryocytic dysplasia, <5% blasts.
(4) Refractory anemia with ringed sideroblasts (RARS)—3% to 11% of all MDS; peripheral smear: anemia, no blasts; bone marrow: unilineage erythroid dysplasia, <5% blasts, 5% ringed sideroblasts.
(5) Refractory cytopenia with multilineage dysplasia (RCMD)—30% of all MDS; peripheral smear: cytopenia(s), <1% blasts, no Auer rods; bone marrow: multilineage dysplasia±ring sideroblasts, <5% blasts, no Auer rods.
(6) Refractory anaemia with excess blasts, type 1 (RAEB-1)—40% of all MDS; peripheral smear: cytopenia(s), <5% blasts, no Auer rods; bone marrow: unilineage or multilineage dysplasia, 5% to 9% blasts, no Auer rods.
(7) Refractory anaemia with excess blasts, type 2 (RAEB-2)—peripheral smear: cytopenia(s), 5% to 19% blasts, ±Auer rods; bone marrow: unilineage or multilineage dysplasia, 10% to 19% blasts, ±Auer rods.
(8) MDS associated with isolated chromosome 5831 deletion (Del 5q)—peripheral smear: anemia, normal or increased platelet count, <1% blasts; bone marrow: isolated chromosome 5831 deletion, hypolobated megakaryocytes, <5% blasts.
(9) Childhood MDS, including refractory cytopenia of childhood (RCC)—peripheral smear: pancytopenia, <5% marrow blasts for RCC, hypocellular marrow.
(10) Myelodysplastic syndrome, unclassified (MDS-U)—peripheral smear: cytopenias, ≦1% blasts, no Auer rods; bone marrow: does not fit any other category, dysplasia or MDS-associated karyotype, <5% blasts, no Auer rods.

In one example, the method of the present disclosure can be used to predict the prognosis of a subject having a MDS classified according to the WHO classification system. For example, a subject diagnosed with a low-risk MDS can be defined as having <5% myeloblasts. Additionally, a subject with 5% myeloblasts can be considered to have advanced or high-risk MDS. It will be appreciated that the method of the present disclosure may additionally or alternatively be used to predict the prognosis of a subject having a MDS classified according to the FAB or IPSS classification systems.

In another aspect, the method of the present disclosure can include obtaining a biological sample from the subject. The biological sample can include a peripheral bodily fluid. For example, the biological sample can comprise fresh blood, stored blood (e.g., in a blood bank), or a blood fraction. The biological sample may be a blood sample expressly obtained for the assay(s) of the present disclosure or, alternatively, a blood sample obtained for another purpose, which can be sub-sampled for the present disclosure. Biological samples can be obtained using standard clinical procedures. Biological samples can be pretreated as necessary by dilution in an appropriate buffer solution, heparinized, concentrated if desired, or fractionated by any number of methods including, but not limited to, ultracentrifugation, fractionation by fast performance liquid chromatography, precipitation with dextran sulfate, or other known methods. Any number of standard aqueous buffer solutions employing one or a combination of buffers, such as phosphate, Tris, or the like, at physiological pH can also be used.

After obtaining the biological sample, another aspect of the present disclosure can include screening or analyzing the biological sample for the presence of at least one predictive parameter that is predictive of the prognosis of a subject suffering from a myeloid malignancy (e.g., MDS or leukemia). As discussed below, the biological sample can be screened or analyzed using any suitable molecular biology technique or assay. In some instances, a predictive parameter can include a mutation in the spliceosomal machinery. In one example, a predictive parameter can include a genetic mutation that results in a dysfunctional, abnormal, and/or modified protein of a spliceosome and, thus, defective splicing. For instance, the genetic mutation can affect a component of the spliceosomal machinery that alters pre-mRNA splicing patterns. In some instances, the genetic mutation can include a point mutation (e.g., a missense or nonsense mutation), an insertion, or a deletion. In other instances, the genetic mutation can be a somatic mutation. In still other instances, the genetic mutation can be heterozygous.

In one aspect, a predictive parameter can include a genetic mutation that results in a dysfunctional, abnormal, and/or modified spliceosome-associated protein, such as splicing factor 3B subunit 1 (SF3B1). In some instances, the genetic mutation in SF3B1 can be a heterozygous somatic mutation. In other instances, the genetic mutation can occur in exon 14 or exon 15 of SF3B1. In one example, the genetic mutation can include a missense mutation that results in a different amino acid residue (e.g., as compared to a germ-line sequence) at position 700 of a SF3B1 protein. For instance, the genetic mutation can result in a KE amino acid change at position 700 of a SF3B1 protein.

In another aspect, a predicative parameter can include a genetic mutation that results in a dysfunctional, abnormal, and/or modified spliceosome-associated protein, such as U2 small nuclear RNA auxiliary factor 1 (U2AF1). In some instances, the genetic mutation in U2AF1 can be a heterozygous somatic mutation. In other instances, the genetic mutation can occur in exon 2 and/or exon 6 of U2AF1. In one example, the genetic mutation can include a missense mutation that results in a different amino acid residue (e.g., as compared to a germ-line sequence) at position 34 of a U2AF1 protein. For instance, the genetic mutation can result in a SF amino acid change at position 34 of a U2AF1 protein. In another example, the genetic mutation can include a missense mutation that results in a different amino acid residue (e.g., as compared to a germ-line sequence) at position 157 of a U2AF1 protein. For instance, the genetic mutation can result in a QP amino acid change at position 157 of a U2AF1 protein.

In another aspect, a predicative parameter can include a genetic mutation that results in a dysfunctional, abnormal, and/or modified spliceosome-associated protein, such as serine/arginine-rich splicing factor 2 (SRSF2). In some instances, the genetic mutation in SRSF2 can be a heterozygous somatic mutation. In other instances, the genetic mutation can include a missense mutation that results in a different amino acid residue (e.g., as compared to a germ-line sequence) at position 95 of a SRSF2 protein. In one example, the genetic mutation can result in a PR amino acid change at position 95 of a SRSF2 protein. In another example, the genetic mutation can result in a PH amino acid change at position 95 of a SRSF2 protein. In yet another example, the genetic mutation can result in a PL amino acid change at position 95 of a SRSF2 protein.

As noted above, one aspect of the present disclosure includes screening or analyzing the biological sample for the presence of at least one predictive parameter using any suitable molecular biology technique or assay. In some instances, a suitable molecular biology technique or assay can include a genetic screening assay (or assays) capable of detecting at least one genetic mutation in the biological sample. Genetic screening assays to detect genetic mutations are known in the art and can include, for example, Sanger sequencing, pyrosequencing, Northern blotting, Southern blotting, and next-generation sequencing (e.g., sequencing by synthesis technology), such as the NGS techniques discussed in the Example below. Other conventional genetic analysis tools, such as DNAnexus software (DNAnexus, Inc., Mountain View, Calif.) can be used in combination with such genetic screening assays to visualize single nucleotide changes, insertions and/or deletions at the gene, exon and base pair levels.

In other instances, a suitable molecular biology technique or assay can include a protein screening assay (or assays) capable of detecting a mutant spliceosome-associated protein. Protein screening assays are known in the art and generally include chemical and/or physical methods for detecting proteins subsequent to their separation. Physical methods are either based on spectroscopy (e.g., light absorption at certain wavelengths) or mass determination of peptides and their fragments using mass spectrometry. Chemical methods are typically used after two-dimensional electrophoresis and employ staining with organic dyes, metal chelates, fluorescent dyes, complexing with silver, or pre-labeling with fluorophores. Western blotting, for example, can be employed by first using gel electrophoresis to separate native proteins by 3-D structure (or denatured proteins by the length of the polypeptide), and then transferring the proteins to a membrane (e.g., nitrocellulose or PVDF), where they are probed using antibodies specific to the target protein. Alternatively or additionally, protein sequencing assays can be employed, such as N-terminal sequencing by Edman degradation.

In another aspect, the presence of at least one predicative parameter in the biological sample can be predictive of the subject's prognosis. In some instances, the presence of at least one somatic mutation in a polynucleotide encoding a spliceosomal-associated protein can be predictive of the subject's prognosis. For example, the prognosis of a subject suffering from a particular myeloid malignancy can be favorable or unfavorable depending upon a particular genetic mutation. A “favorable prognosis” can refer to an increased likelihood that a subject with a particular myeloid malignancy will experience longer survival as compared to a subject without the same genetic mutation and with the same myeloid malignancy. An “unfavorable prognosis” can refer to a decreased likelihood that a subject with a particular myeloid malignancy will experience shorter survival as compared to a subject without the same genetic mutation with the same myeloid malignancy.

In one example, a detected somatic mutation in SF3B1 may be indicative of a favorable prognosis in a subject suffering from low-risk MDS, such as RARS. For instance, a somatic mutation that results in an amino acid substitution at position 700 of a SF3B1 protein (e.g., K700E) may be indicative of a favorable prognosis in a subject suffering from a low-risk MDS.

In another example, a detected somaticd mutation in U2AF1 may be indicative of an unfavorable prognosis in a subject suffering from a high-risk MDS or leukemia (e.g., CMML, sAML). For instance, a somatic mutation that results in an amino acid substitution at position 34 of a U2AF1 protein (e.g., S34F) may be indicative of an unfavorable prognosis in a subject suffering from a high-risk MDS or leukemia. Additionally or alternatively, a somatic mutation that results in an amino acid substitution at position 157 of a U2AF1 protein (e.g., Q157P) may be indicative of an unfavorable prognosis in a subject suffering from a high-risk MDS or leukemia.

In another example, a detected somatic mutation in SRSF2 may be indicative of an unfavorable prognosis in a subject suffering from a low-risk MDS. For instance, a somatic mutation that results in an amino acid substitution at position 95 of a SRSF2 protein (e.g., P95H, P95R, P95L) may be indicative of an unfavorable prognosis in a subject suffering from a low-risk MDS.

In another aspect, a new or more aggressive treatment regimen can be implemented in a subject having a MDS or leukemia, for example, and being diagnosed with an unfavorable prognosis. Standard care for MDS typically includes supportive therapy, including transfusions, and may include bone marrow stimulation and cytotoxic chemotherapy. Thus, depending upon the particular myeloid malignancy affecting the subject, a new or more aggressive treatment regimen can include increasing the subject's current medication dosage(s), treatment with additional medication(s), and/or discontinuing treatment with current medication(s) and initiating treatment with new medication(s), as well as various surgical approaches (e.g., bone marrow transplantation).

Another aspect of the present disclosure includes a method for diagnosing a subject with a high-risk MDS or leukemia (e.g., CMML, sAML refractory anemia with excess blasts or RAEB). The method can include obtaining a biological sample from the subject (as described above). After obtaining the biological sample, the sample can be screened or analyzed for the presence of at least one mutation in a SRSF2 protein, or a polynucleotide encoding the SRSF2 protein that results in defective splicing. Examples of conventional screening and detection techniques are described above.

In some instances, the genetic mutation in SRSF2 can be a heterozygous somatic mutation. In other instances, the genetic mutation can include a missense mutation that results in a different amino acid residue at position 95 of a SRSF2 protein. In one example, the genetic mutation can result in a PR amino acid change at position 95 of a SRSF2 protein. In another example, the genetic mutation can result in a PH amino acid change at position 95 of a SRSF2 protein. In yet another example, the genetic mutation can result in a PL amino acid change at position 95 of a SRSF2 protein.

In another aspect, the presence of at least one mutation in a SRSF2 protein, or a polynucleotide encoding the SRSF2 protein that results in defective splicing can be indicative of a high risk MDS or leukemia in the subject. In one example, a detected somatic mutation in SRSF2 may be indicative of a high-risk MDS or leukemia in the subject. For instance, a somatic mutation that results in an amino acid substitution at position 95 of a SRSF2 protein (e.g., P95H, P95R, P95L) may be indicative of a high-risk MDS or leukemia in the subject.

Following diagnosis of the subject with a high-risk MDS or leukemia, a new or more aggressive treatment regimen can be implemented (as discussed above).

The following example is for the purpose of illustration only and is not intended to limit the scope of the claims, which are appended hereto.

### Example

Methods

Patient Population

Bone marrow aspirates or blood samples were collected from 310 patients with MDS (N=87), MDS/myeloproliferative neoplasms (MDS/MPN, N=63), MPN (N=51), secondary AML (sAML) (N=54) that evolved from these conditions and primary AML (pAML) (N=55) seen at Cleveland Clinic between 2003 and 2008 (Table 1).

TABLE 1
Clinical characteristics of patients participating in this study

| | |
|---|---|
| MDS | 87 |
| Low risk | 57 |
| RCUD/RCMD/5q-/MDS-U | 37 |
| RARS | 20 |
| High risk | |
| RAEB ½ | 30 |
| MDS/MPN | 63 |
| CMML/aCML | 46 |
| MDS/MPN-U (RARS-T) | 17 (11) |
| MPN | 51 |
| PV/PMF/ET | 15 |
| CML | 36 |
| AML | 109 |
| Primary AML | 55 |
| Secondary AML | 54 |

* includes 8 cases with therapy related myeloid malignancies.
MDS, myelodysplastic syndromes; RCUD, refractory cytopenia with unilineage dysplasia; RCMD, refractory cytopenia with multilineage dysplasia; MDS-U, MDS unclassifiable; RARS, refractory anemia with ring sideroblasts; RAEB, refractory anemia with excess blasts; MDS/MPN, MDS/myeloproliferative neoplasms; CMML, chronic myelomonocytic leukemia; aCML, atypical chronic myelogenous leukemia; RARS-T, RARS associated with marked thrombocytosis; PV, polycythemia vera; PMF, primary myelofibrosis; ET, essential thrombocytopenia; AML, acute myeloid leukemia.

Informed consent for sample collection was obtained according to a protocol approved by the institutional IRB and in accordance with the Declaration of Helsinki. Diagnosis was confirmed and assigned according to World Health Organization (WHO) classification criteria. Low-risk MDS was defined as patients having <5% myeloblasts. Patients with ≧5% myeloblasts constituted those with advanced disease. Serial samples were obtained for 38 patients. To study the germline genotype, immunoselected CD3+ lymphocytes were used. Cytogenetic analysis was performed according to standard banding techniques based on 20 metaphases. Clinical parameters studied included age, sex, overall survival, blood counts, and metaphase cytogenetics. The median follow up of the cohort was 18 months (1-168 months).

Cytogenetics and Single Nucleotide

Polymorphism Array (SNP-A) Analyses

Technical details regarding sample processing for SNP-A assays were previously described (Maciejewski, J. P. et al., Br J Haematol 146, 479-88, 2009; Gondek, L. P. et al., Blood 111, 1534-42 (2008). Affymetrix 250K and 6.0 Kit (Affymetrix, Santa Clara, Calif.) were used. A stringent algorithm was applied in the identification of SNP-A lesions. Patients with SNP-A lesions concordant with metaphase cytogenetics or typical lesions known to be recurrent required no further analysis. Changes reported in our internal or publicly-available (Database of Genomic Variants) copy number variation (CNV) databases were considered non-somatic and excluded. Results were analyzed using CNAG (v3.0) or Genotyping Console (Affymetrix). All other lesions were confirmed as somatic or germline by analysis of CD3-sorted cells.

Whole Exome Sequencing

Genomic DNA was extracted from bone marrow or peripheral blood using standard methods and subjected agarose gel and OD ratio tests to confirm the purity and concentration prior to Covaris (Covaris, Inc., Woburn, Mass.) fragmentation. 0.5-2.5 μg of fragmented genomic DNA was tested for size distribution and concentration using an Agilent Bioanalyzer 2100 and Nanodrop. Illumina libraries were made from qualified fragmented gDNA using NEBNext reagents (New England Biolabs, Ipswich, Mass.) and the resulting libraries were subjected to exome enrichment using NimbleGen SeqCap EZ Human Exome Library v2.0 (Roche NimbleGen, Inc., Madison, Wis.) following the manufacturer's instructions. Enriched libraries were tested for enrichment by qPCR and for size distribution and concentration by an Agilent Bioanalyzer 2100. The samples were then sequenced on an Illumina HiSeq2000 which generated paired-end reads of 100 nucleotides. Paired bone marrow mononuclear cells and CD3+ peripheral blood lymphocytes were used as germline controls. DNAnexus software (DNAnexus, Inc, Mountain View, Calif.) was used to visualize single nucleotide changes, insertions and/or deletions at the gene, exon and base pair levels. A rational bioanalytic algorithm was applied to identify candidate non-synonymous alterations. Multiple steps were performed to reduce the false positive rate within reported results. First, whole exome assembly was non-redundantly mapped using the reference genome hg19. Next, the analytic algorithm within DNAnexus called all the positions that vary from a reference genome. Each potential mutation was compared against databases of known SNPs, including Entrez Gene and the Ensembl Genome Browser. These candidate alterations were subtracted by the results of CD3+ peripheral blood DNA and subsequently validated using Sanger sequencing (see below). Moreover, spliceosome-associated gene mutations were screened using whole exome sequencing results available through The Cancer Genome Atras (TOGA).

Sanger Sequencing Analysis

All exons of the selected genes were amplified and underwent direct genomic sequencing by standard techniques on the ABI 3730×1 DNA analyzer (Applied Biosystems, Foster City, Calif.) as previously described (Dunbar, A. J. et al., Cancer Res 68, 10349-57, 2008; Jankowska, A. M. et al., Blood 113, 6403-10, 2009; Makishima, H. et al., Blood 117, e198-206, 2011). All mutations were detected by bidirectional sequencing and scored as pathogenic if not present in non-clonal paired CD3-derived DNA. Frame shift mutations were validated by cloning and sequencing individual colonies (TOPO TA cloning, Invitrogen, Carlsbad, Calif.). For confirmation of the somatic nature of the mutations, exons containing mutations were tested in non-clonal control DNA.

Whole RNA Deep Sequencing

Total RNA was extracted from bone marrow mononuclear cells using the Nucleospin RNA II Kit (Macherey-Nagel, Bethlehem, Pa.) with DNAase treatment. The integrity and purity of total RNA were assessed using Agilent Bioanalyzer and OD260/280. 1-2 μg of cDNA was generated using Clontech SmartPCR cDNA kit (Clontech Laboratories, Inc., Mountain View, Calif.) from 100 ng of total RNA. cDNA was fragmented using Covaris (Covaris, Inc., Woburn, Mass.), profiled using Agilent Bioanalyzer, and subjected to Illumina library preparation using NEBNext reagents (New England Biolabs, Ipswich, Mass.). The quality and quantity and the size distribution of the Illumina libraries were determined using an Agilent Bioanalyzer 2100. The libraries were then submitted for Illumina HiSeq2000 sequencing according to the standard operation. Paired-end 90 base pair reads were generated and subjected to data analysis using the platform provided by DNAnexus. DNAnexus software allowed visualization of reads derived from spliced mRNA and those that completely match the genome, including both sense and antisense.

Statistical Analysis of Clinical Data

The Kaplan-Meier method was used to analyze survival outcomes (overall survival) of subgroups characterized by the presence of mutant vs. wild type variants of specific spliceosome-associated gene mutations with the log-rank test and Wilcoxon test (JMP9; SAS, Cary, N.C.). Significance was determined at a two-sided alpha level of 0.05.

Results

Detection of Somatic Spliceosomal Mutations in Myeloid Malignancies

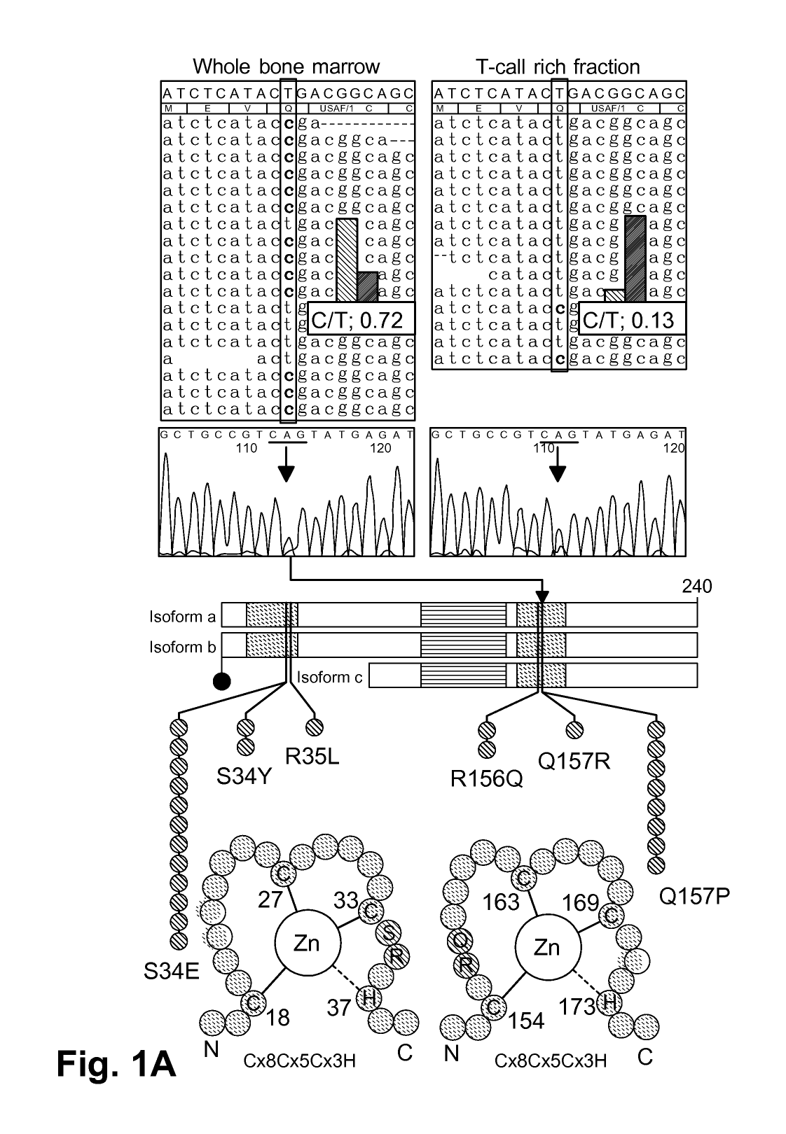

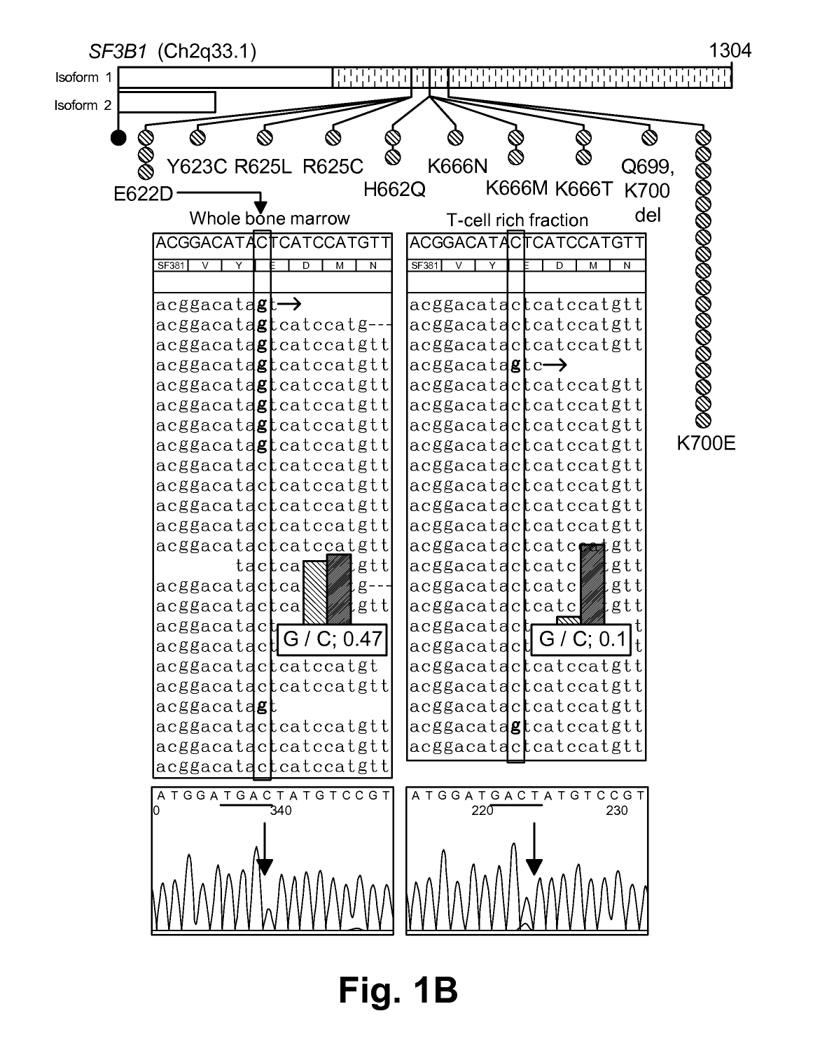

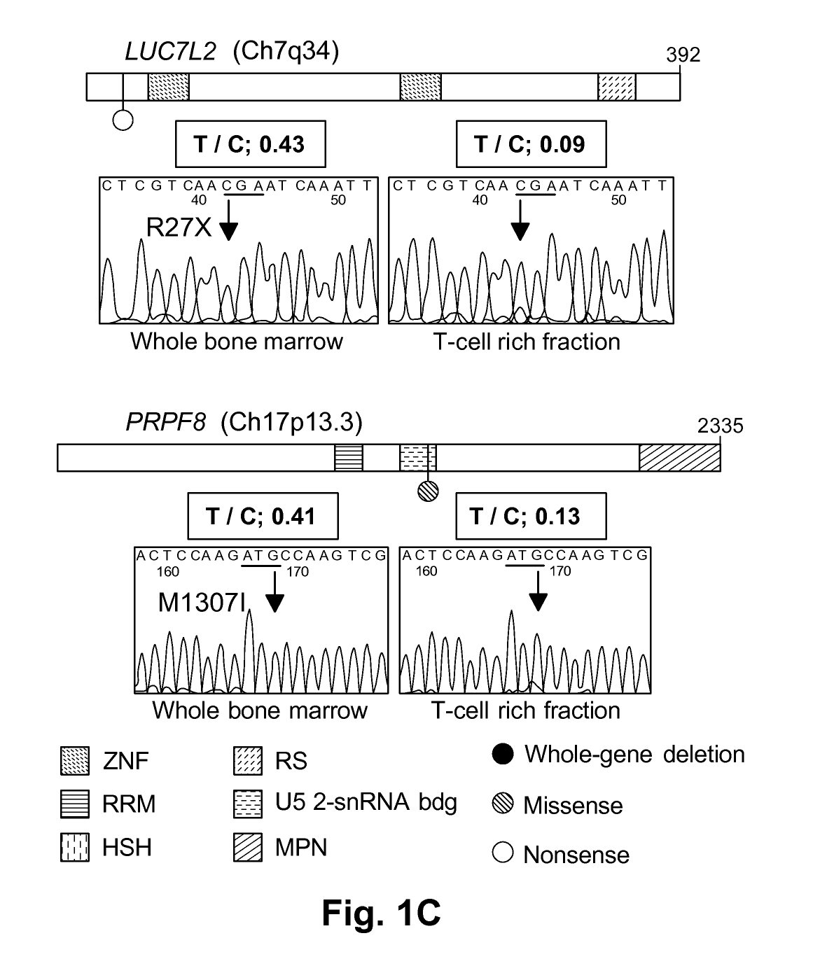

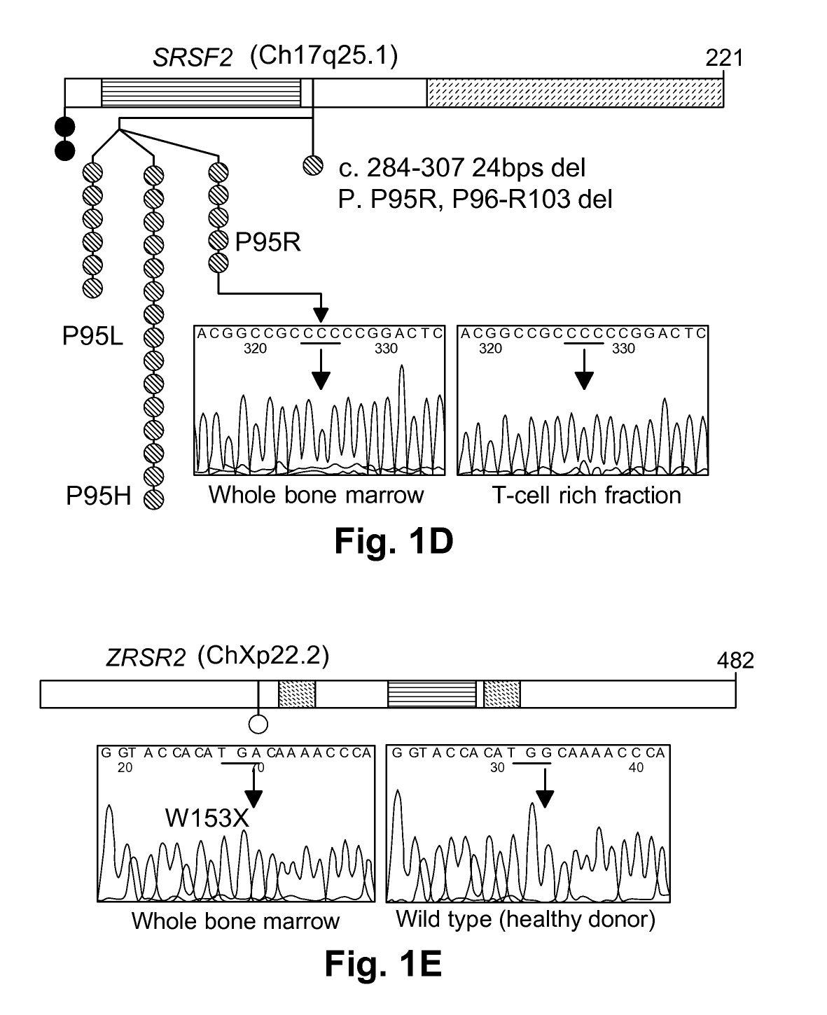

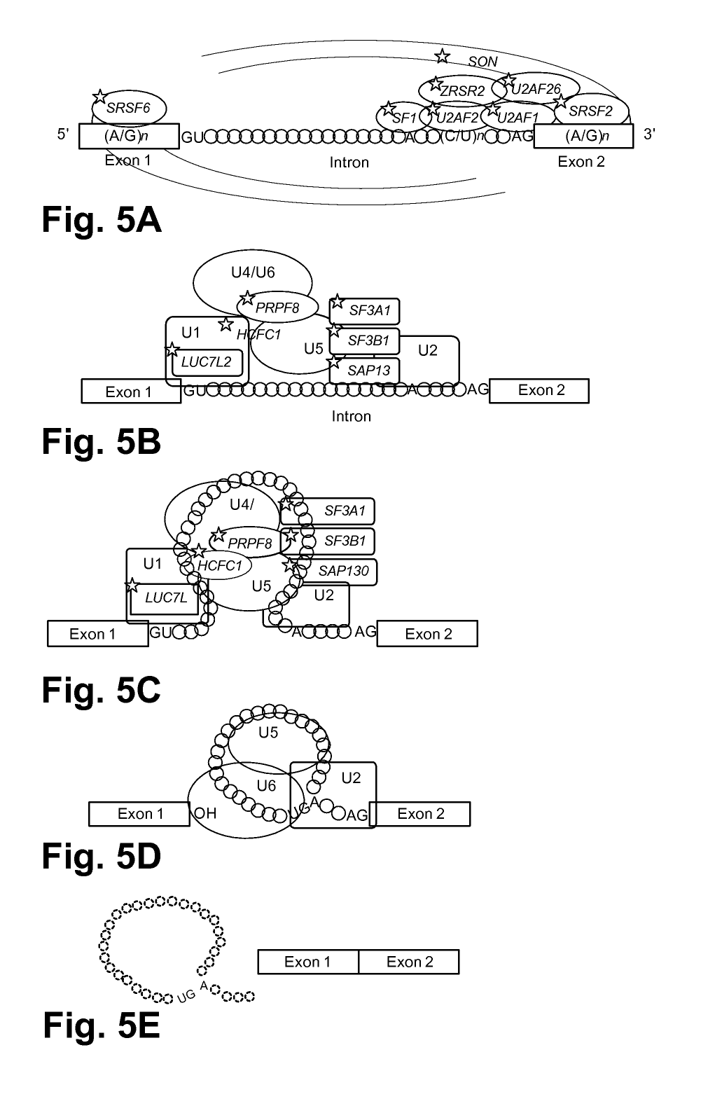

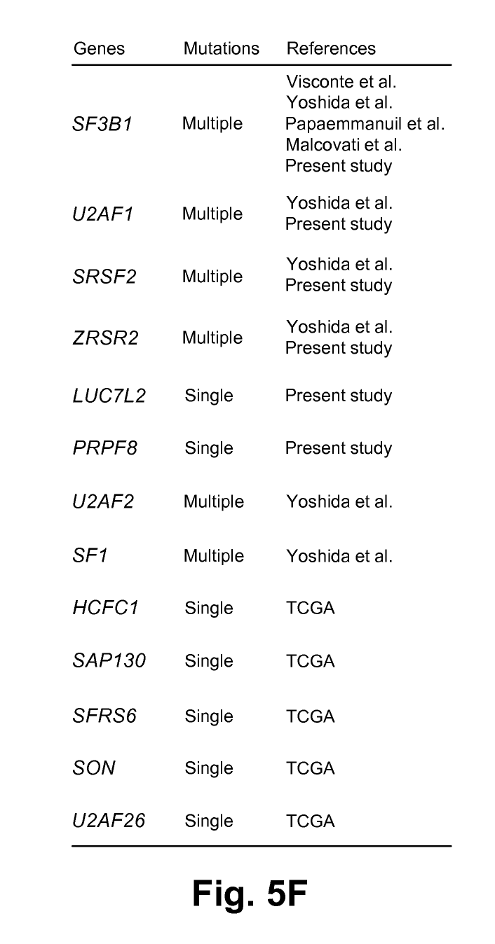

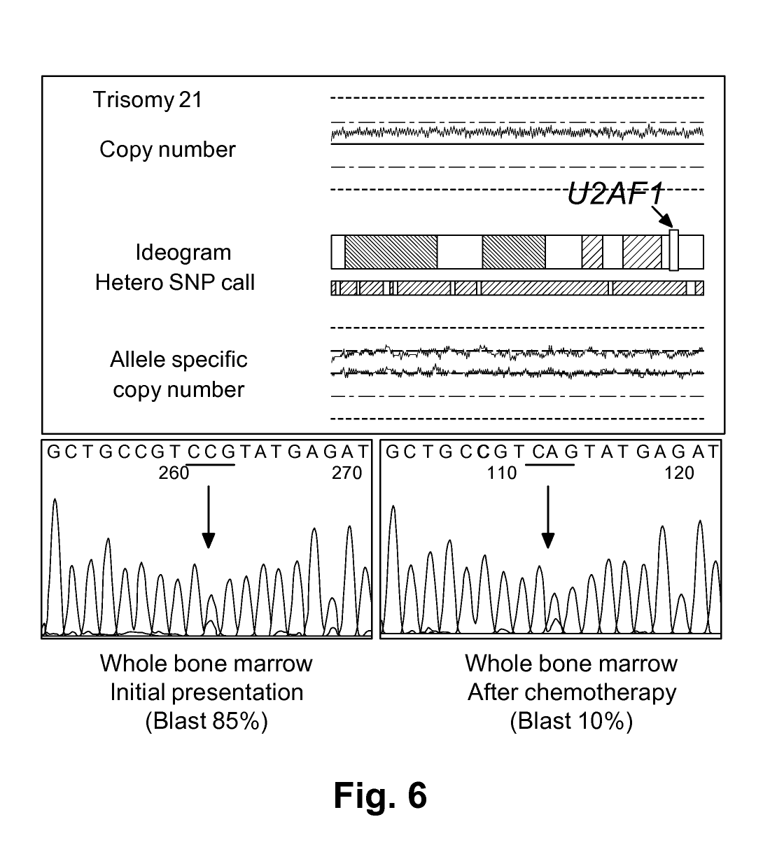

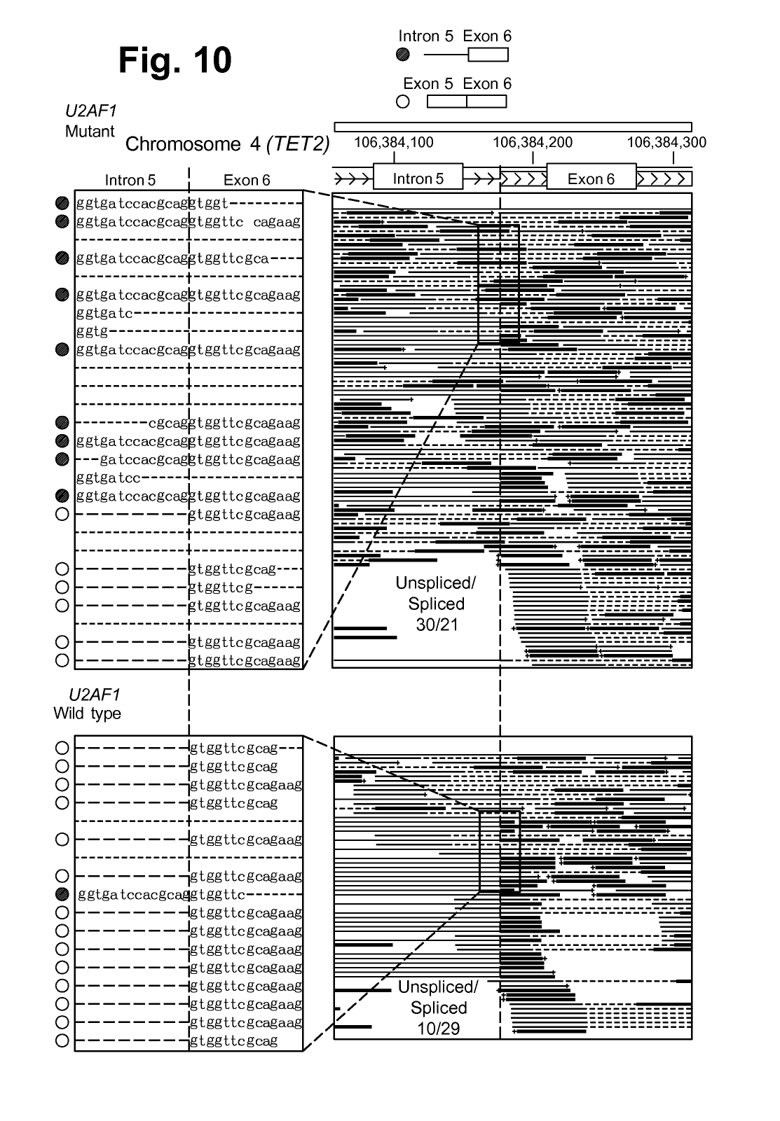

Initially, we performed whole exome sequencing of 15 index cases with various forms of chronic myeloid malignancies and identified distinct somatic mutations in genes encoding components of the spliceosomal machinery. A heterozygous missense mutation in U2AF1 (Q157R) was found in a patient with sAML and UPD2q (FIG. 1A) as well as in another sAML patient with trisomy 21 (FIG. 6). Similarly, heterozygous, somatic SF3B1 mutations (E622D) were detected in a patient with refractory anemia with ring sideroblasts (RARS) associated with marked thrombocytosis (RARS-t) (FIG. 1B). In two patients with sAML, we identified a somatic mutation (M13071) in PRPF8 and a heterozygous mutation (R27X) in LUC7L2, respectively (FIG. 10). Based on these findings, we screened other genes within the spliceosomal machinery in patients with MDS and related disorders (N=120). Using this targeted approach, we identified mutations in ZRSR2 (W153X) and SRSF2 (P95R) (FIGS. 1D-E). All these mutations were somatic as confirmed by sequencing of the corresponding germline-derived DNA. Moreover, screening of the whole exome sequencing results for AML available through the Cancer Genome Atlas revealed the presence of mutations in genes encoding other components of the spliceosome, including mutations in HCFC1 (P72L), SAP130 (T2471), SRSF6 (W123S), SON (M1024V) and U2AF26 (C116X) (FIG. 5). We found that among the initially screened cohort of patients with myeloid malignancies, mutations were highly recurrent in U2AF1, SF3B1 and SRSF2, while mutations of other spliceosomal genes were not detected, indicating a frequency of <1%. While we identified 2 mutant copies of U2AF1 (21q22.3) in a sAML case with trisomy 21, all other 19 cases of trisomy 21 screened for this mutation were negative. Moreover, no homozygous mutations were found in patients with UPD21q (N=8). Similarly, all mutations of SF3B1 and SRSF2 were heterozygous, and patients with somatic UPD2q33.1 or UPD17q25 (regions containing SF3B1 and SRSF2, respectively) did not harbor homozygous mutations of the associated genes.

Clinical Associations and Frequencies of

Spliceosomal Mutations in Myeloid Malignancies

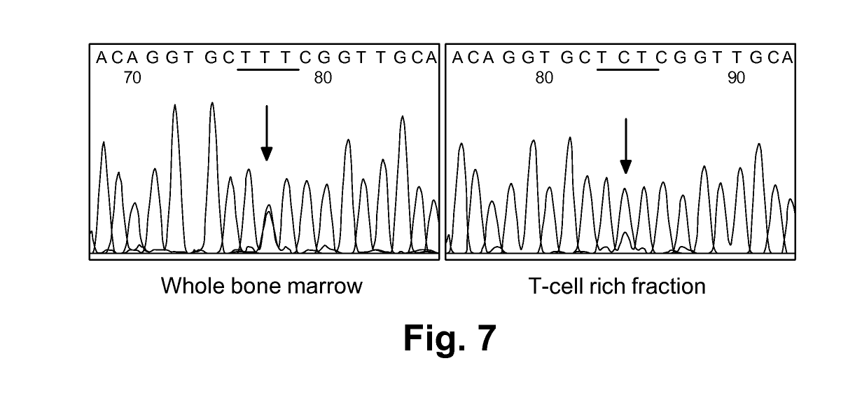

We subsequently screened a large cohort of patients (N=310) with MDS and related disorders in a stepwise fashion to determine the frequency of spliceosomal mutations discovered in the index cases. Based on the initial screen (N=120; see above), we noted that mutations in U2AF1, SF3B1 and SRSF2 were the most frequent. All SF3B1 mutations were located in exon 14 or 15, with the K700 mutation being the most recurrent (FIG. 1B). Similarly, all mutations in SRSF2 affected position P95 (FIG. 1D). In contrast, mutations in U2AF1 affected exons 2 and 6, corresponding to the 2 zinc finger domains of this protein (FIG. 1A and FIG. 7). The extended cohort of patients was used to identify phenotype/genotype associations (see Appendix A, which lists the clinical characteristics of individual patients affected by spliceosomal mutations, as well as Appendix B, which lists clinical characteristics of individual patients affected by spliceosomal mutations).

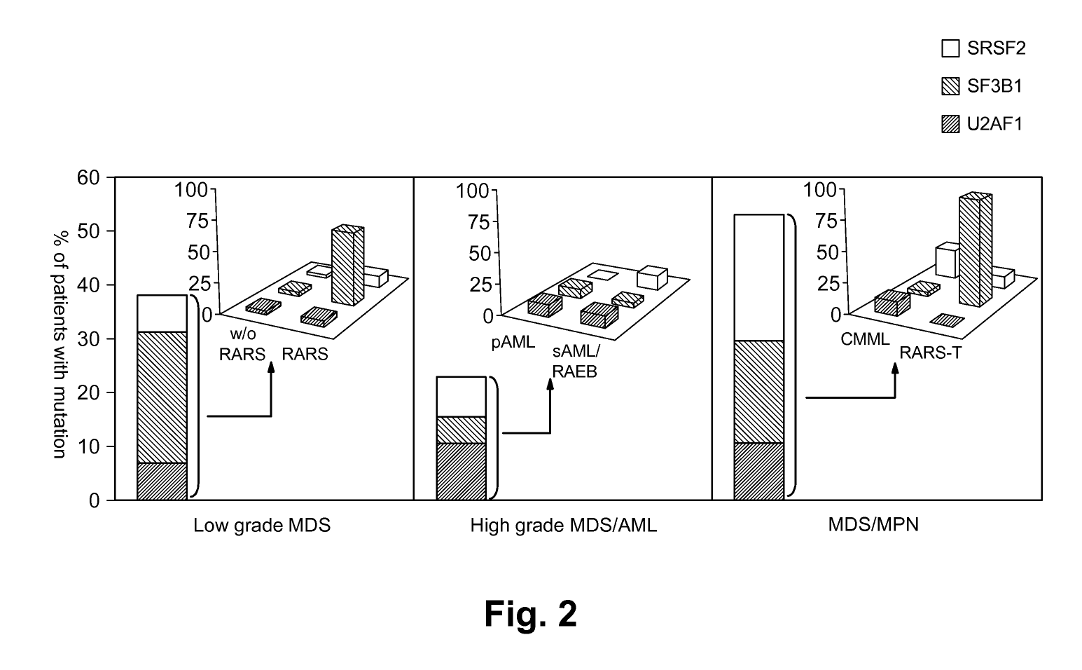

In low-risk MDS, mutations of any one of these three genes were found in 39% of patients, and further analysis revealed that mutations in SF3B1 were highly associated with RARS. Among patients with MDS/MPN, SF3B1 mutations were not common in CMML, but they were frequent in patients with RARS-t and thus the presence of RS was found to correlate highly with SF3B1 mutations, irrespective of other clinical or morphologic features (FIG. 2). In contrast, U2AF1 mutations are most frequent in the high risk MDS/AML cohort (11%), while SRSF2 was most frequently mutated in MDS/MPN (24%) particularly in CMML (28%) (FIG. 2).

Impact of Spliceosomal Mutations on Clinical Outcomes

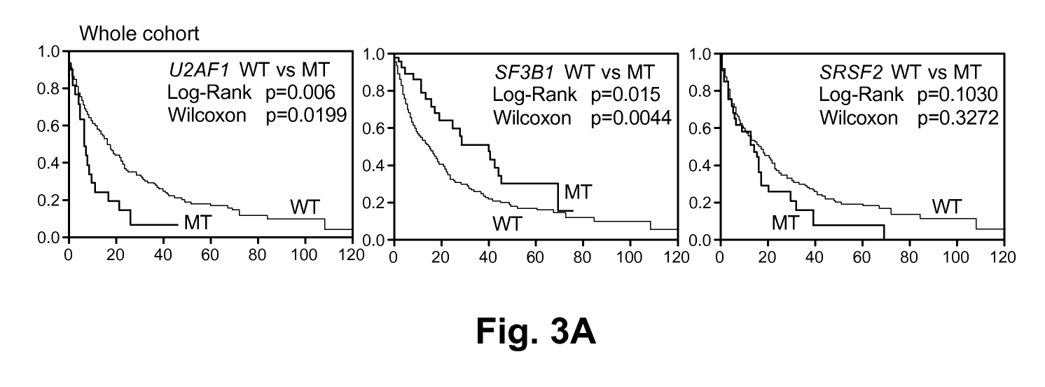

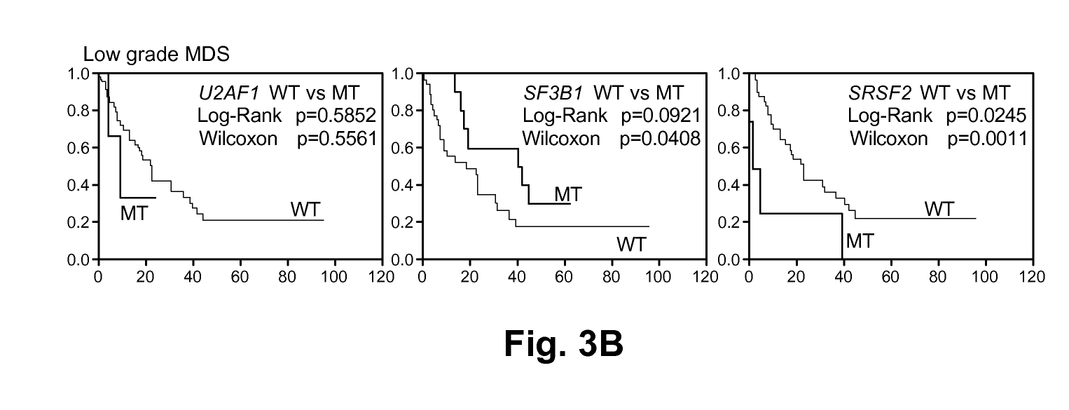

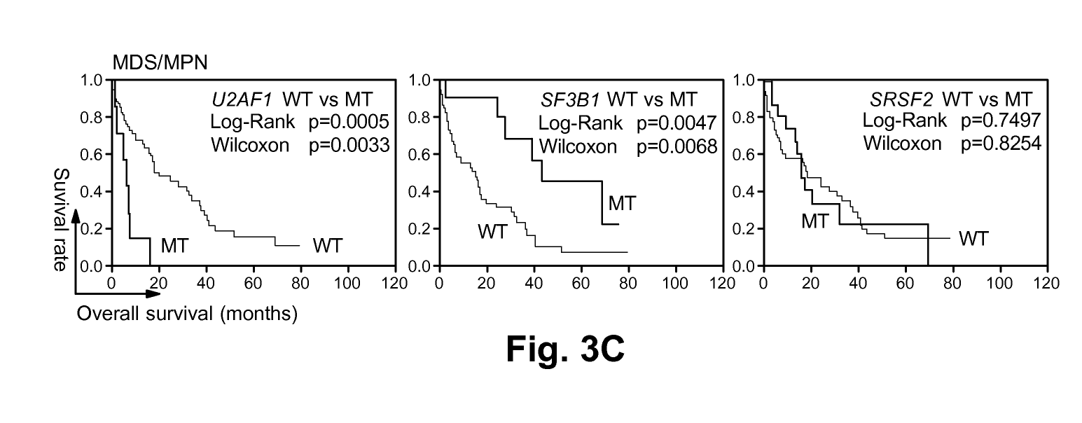

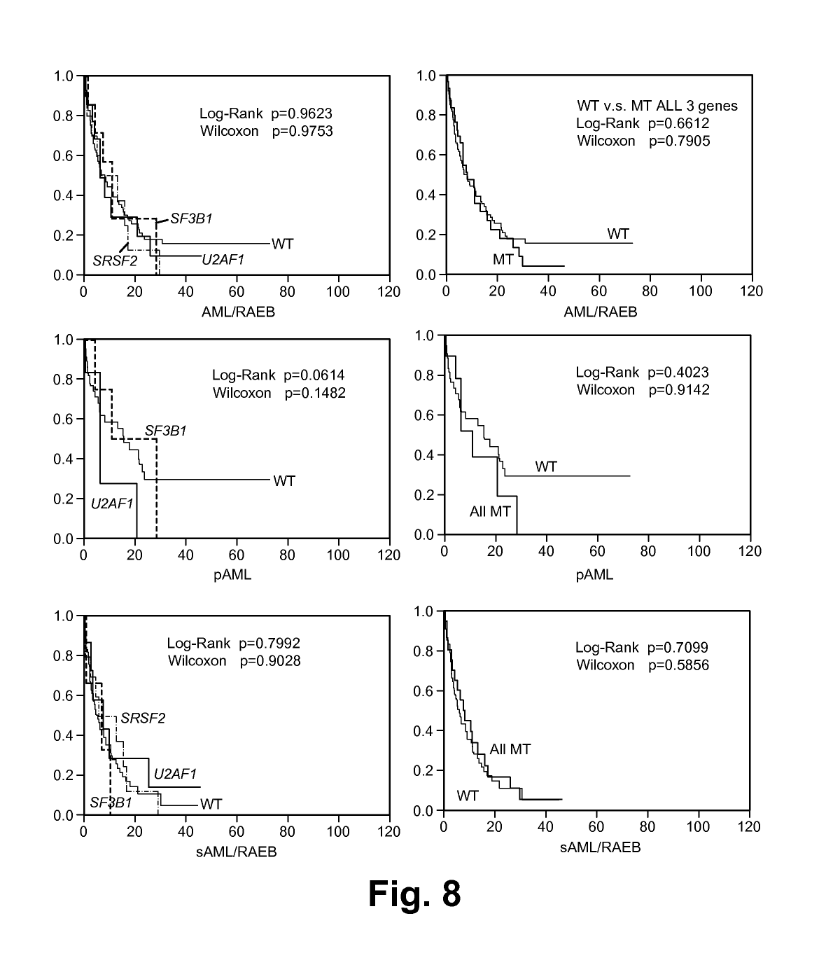

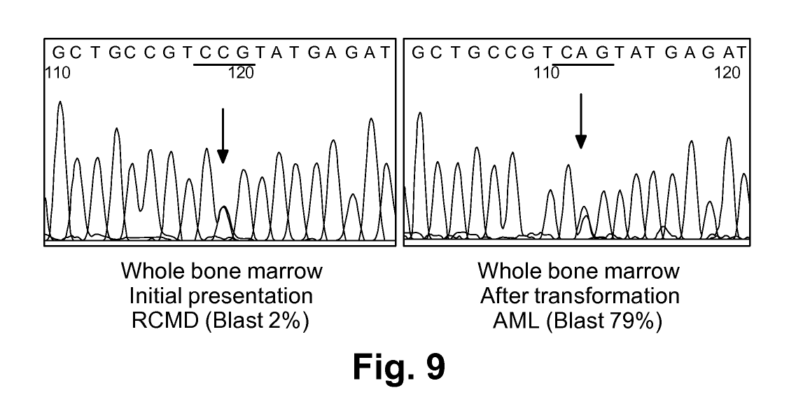

Subsequently, we studied the impact of the most common spliceosomal mutations on clinical outcomes. We first analyzed the entire cohort of patients (Table 1) and determined the survival of patients in whom the three most common spliceosomal mutations were present. When 310 patients genotyped for these mutations were analyzed (Table 1), the presence of SF3B1 mutations was associated with longer survival, U2AF1 mutations with shorter survival, while SRSF2 mutations had no effect on survival. We then analyzed the impact of these mutations in more clinically uniform subgroups to more precisely determine their clinical consequences. As expected, in sAML and pAML, due to overall poor prognosis, the presence of spliceosomal mutations did not further affect survival (FIG. 8). However, in low-risk MDS, patients with SF3B1 mutations showed better prognosis, while those with SRSF2 mutations had worse survival. In MDS/MPN, SRSF2 mutations were more common than U2AF1; however, U2AF1 mutations were associated with shorter survival (FIG. 3). When serial samples were analyzed, we found that U2AF1 (FIGS. 5-6) and SF3B1 (data not shown) mutations detected in the sAML stage were present from initial MDS presentations, suggesting an ancestral origin of this mutation. Overall, SF3B1 mutations were less prevalent in patients with advanced forms of MDS, indicating that mutation of this factor does not contribute to progression (FIG. 2).

Effects of Spliceosomal Mutations on Spliceosomal Function

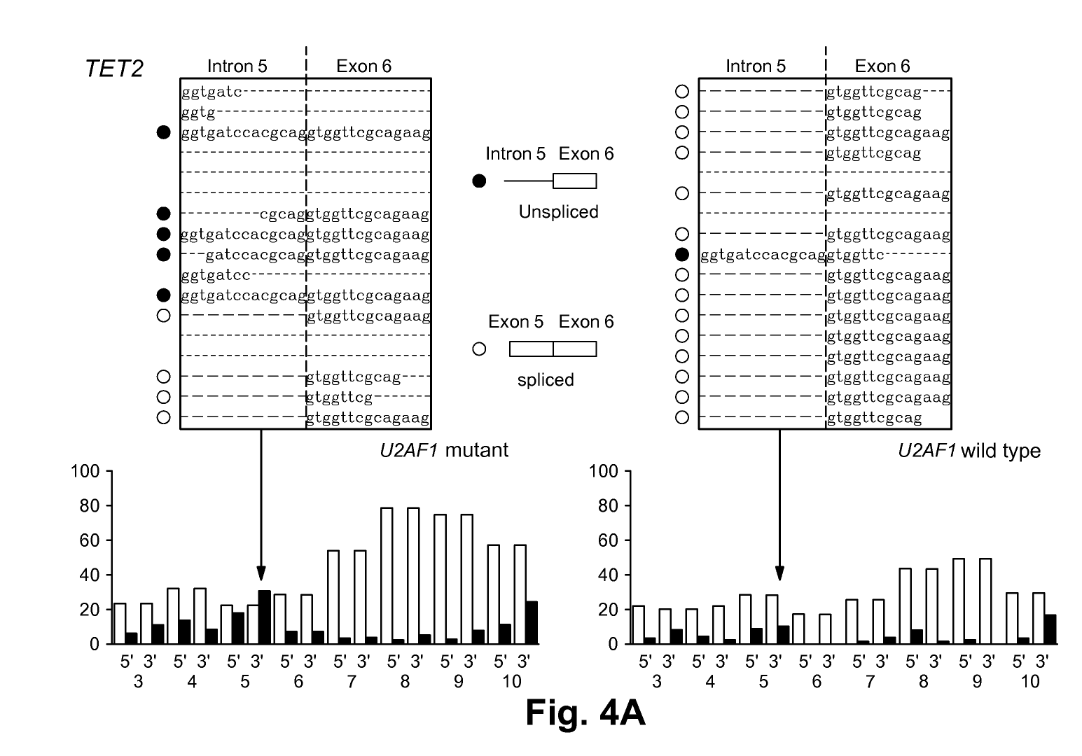

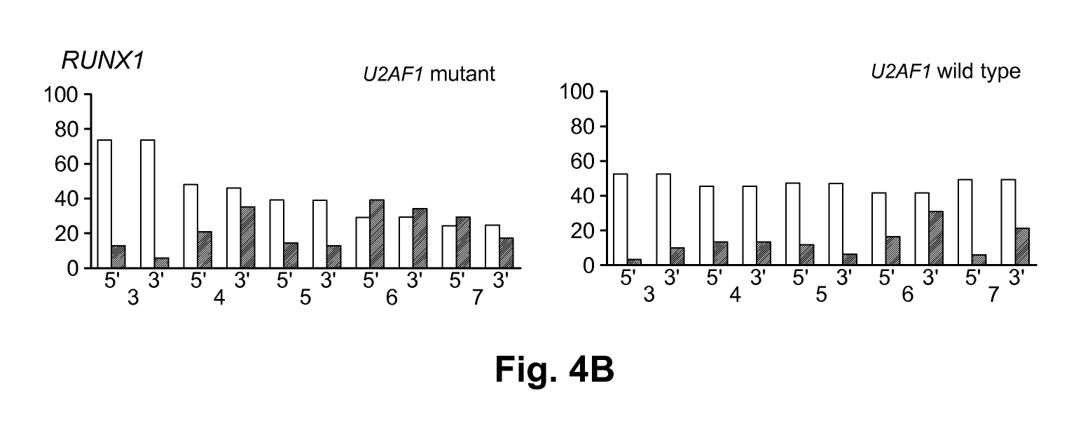

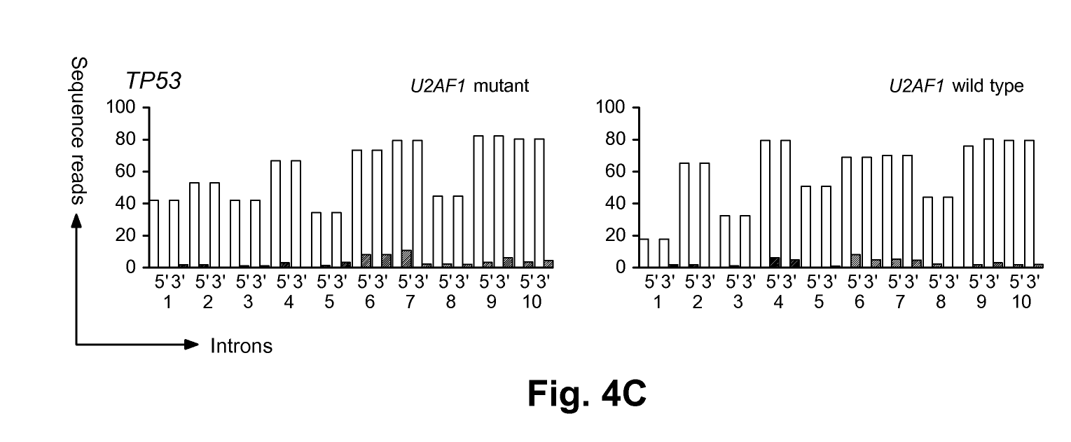

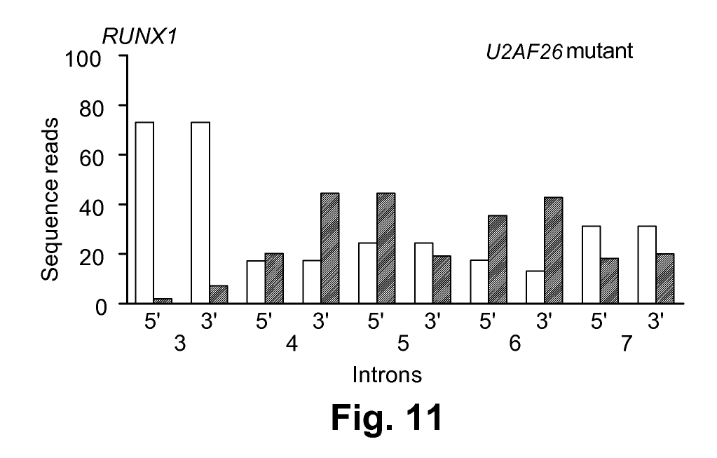

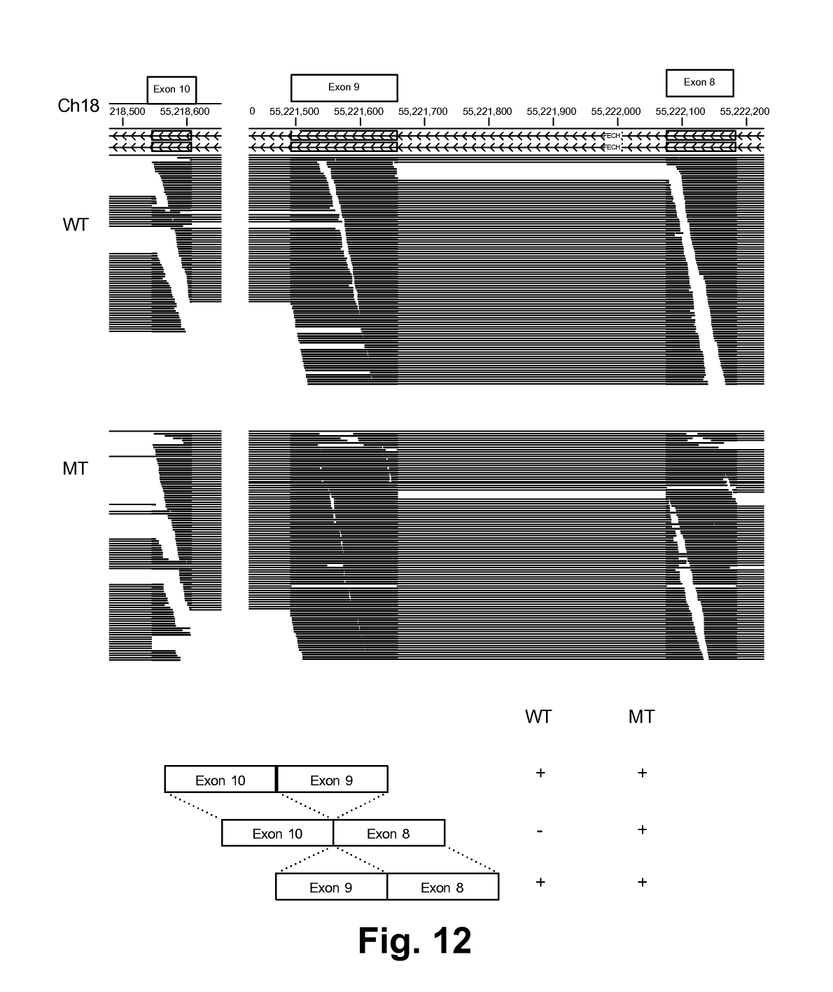

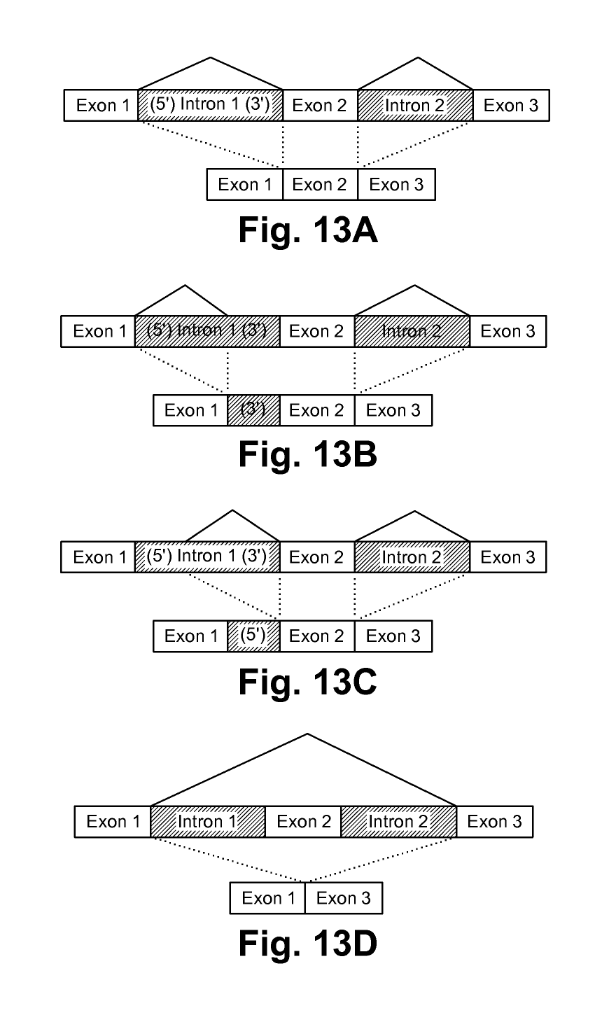

Conceptually, mutations of spliceosomal proteins could result in defective splicing, including intron retention, altered splice site recognition or altered alternative splicing. To determine the functional consequences of spliceosomal mutations on splicing, we performed whole mRNA deep sequencing. In the presence of a functional spliceosomal machinery, sequencing reads are expected to not cross the intron/exon boundaries, and therefore should not contain any intronic sequences. We analyzed RNA sequencing results in patients with mutations in U2AF1 (N=3), SF3B1 (N=2) and U2AF26 (N=1), as well as in a healthy control and one MDS patient with a wild type configuration of these genes. There was no genome-wide increase in intron retention observed in the mutant patients. However, we found a number of specific genes in which the splicing pattern was altered. For instance, U2AF1 mutations were associated with defective splicing of intron 5 of TET2 at both splice sites (FIG. 4A and FIG. 10), while splicing of other TET2 introns were less affected. Another gene in which splicing was affected was RUNX1 (FIG. 4B). In contrast, TP53 splicing was unaffected (FIG. 4C). Similarly, U2AF26 mutations resulted in an alteration of RUNX1 splicing (FIG. 11). Moreover, alternative splicing analysis showed that exon 9 of FECH was skipped in U2AF1 mutant cases but not in U2AF1 WT cases, including those with SF3B1 mutations (FIG. 12).

From the above description of the present disclosure, those skilled in the art will perceive improvements, changes and modifications. Such improvements, changes, and modifications are within the skill of the art and are intended to be covered by the appended claims.

Having described the invention, the following is claimed:

1. A method for predicting the prognosis of a subject with a myeloid malignancy, said method comprising:
obtaining a biological sample from the subject; and
detecting, in the biological sample, at least one mutation in a spliceosome-associated protein, or a polynucleotide encoding the spliceosome-associated protein that results in defective splicing;
wherein the presence of at least one mutation in the spliceosome-associated protein, or a polynucleotide encoding the spliceosome-associated protein, is indicative of the subject's prognosis.

2. The method of claim 1, wherein the polynucleotide encoding a spliceosome-associated protein is selected from the group consisting of a SF3B1 gene, a U2AF1 gene, and a SRSF2 gene.

3. The method of claim 2, wherein a detected somatic mutation in the SF3B1 gene is indicative of a favorable prognosis in a subject suffering from a low-risk myelodysplastic syndrome (MDS).

4. The method of claim 3, wherein the somatic mutation results in an amino acid substitution at position 700 of a SF3B1 protein.

5. The method of claim 2, wherein a detected somatic mutation in a U2AF1 gene is indicative of an unfavorable prognosis in a subject suffering from a high-risk MDS or leukemia.

6. The method of claim 5, wherein the somatic mutation results in an amino acid substitution at position 34 of a U2AF1 protein.

7. The method of claim 5, wherein the somatic mutation results in an amino acid substitution at position 157 of a U2AF1 protein.

8. The method of claim 2, wherein a detected somatic mutation in a SRSF2 gene is indicative of an unfavorable prognosis in a subject suffering from a low-risk MDS.

9. The method of claim 8, wherein the somatic mutation results in an amino acid substitution at position 95 of a SRSF2 protein.

10. A method for treating a patient with a myeloid malignancy, said method comprising the steps of:
obtaining a biological sample from the subject;
detecting, in the biological sample, at least one mutation in a spliceosome-associated protein, or a polynucleotide encoding the spliceosome-associated protein that results in defective splicing; and
administering a treatment regimen to a subject having the at least one mutation in a spliceosome-associated protein, or a polynucleotide encoding the spliceosome-associated protein.

11. The method of claim 10, wherein the at least one mutation is a somatic mutation in a SRSF2 gene or a U2AF1 gene.

12. The method of claim 11, wherein the somatic mutation in the U2AF1 gene results in an amino acid substitution at position 34 of a U2AF1 protein.

13. The method of claim 11, wherein the somatic mutation in the U2AF1 gene results in an amino acid substitution at position 157 of a U2AF1 protein.

14. The method of claim 11, wherein the somatic mutation in the SRSF2 gene results in an amino acid substitution at position 95 of a SRSF2 protein.


SEQUENCE LISTING (48 sequences)

SEQ ID NO: 1 - DNA, 19 bp, Homo sapiens
atctcatactgacggcagc

SEQ ID NO: 2 - protein, 4 aa, Homo sapiens
QYEM

SEQ ID NO: 3 - DNA, 12 bp, Homo sapiens
atctcataccga

SEQ ID NO: 4 - DNA, 17 bp, Homo sapiens
atctcataccgacggca

SEQ ID NO: 5 - DNA, 19 bp, Homo sapiens
atctcataccgacggcagc

SEQ ID NO: 6 - DNA, 19 bp, Homo sapiens
  modified_base (15)..(15): a, c, t, g, unknown or other
atctcatactgacgncagc

SEQ ID NO: 7 - DNA, 19 bp, Homo sapiens
  modified_base (15)..(15): a, c, t, g, unknown or other
atctcataccgacgncagc

SEQ ID NO: 8 - DNA, 19 bp, Homo sapiens
  modified_base (15)..(16): a, c, t, g, unknown or other
atctcataccgacgnnagc

SEQ ID NO: 9 - DNA, 19 bp, Homo sapiens
  modified_base (11)..(19): a, c, t, g, unknown or other
atctcatactnnnnnnnnn

SEQ ID NO: 10 - DNA, 13 bp, Homo sapiens
aactgacggcagc

SEQ ID NO: 11 - DNA, 19 bp, Homo sapiens
gctgccgtcagtatgagat

SEQ ID NO: 12 - DNA, 19 bp, Homo sapiens
  modified_base (16)..(16): a, c, t, g, unknown or other
atctcatactgacggnagc

SEQ ID NO: 13 - DNA, 18 bp, Homo sapiens
  modified_base (15)..(15): a, c, t, g, unknown or other
tctcatactgacggnagc

SEQ ID NO: 14 - DNA, 15 bp, Homo sapiens
  modified_base (12)..(12): a, c, t, g, unknown or other
catactgacggnagc

SEQ ID NO: 15 - DNA, 19 bp, Homo sapiens
  modified_base (14)..(16): a, c, t, g, unknown or other
atctcatactgacnnnagc

SEQ ID NO: 16 - DNA, 19 bp, Homo sapiens
  modified_base (11)..(19): a, c, t, g, unknown or other
atctcataccnnnnnnnnn

SEQ ID NO: 17 - DNA, 21 bp, Homo sapiens
acggacatactcatccatgtt

SEQ ID NO: 18 - protein, 6 aa, Homo sapiens
NMDEYV

SEQ ID NO: 19 - DNA, 11 bp, Homo sapiens
acggacatagt

SEQ ID NO: 20 - DNA, 19 bp, Homo sapiens
acggacatagtcatccatg

SEQ ID NO: 21 - DNA, 21 bp, Homo sapiens
acggacatagtcatccatgtt

SEQ ID NO: 22 - DNA, 14 bp, Homo sapiens
  modified_base (7)..(11): a, c, t, g, unknown or other
tactcannnnngtt

SEQ ID NO: 23 - DNA, 19 bp, Homo sapiens
  modified_base (14)..(18): a, c, t, g, unknown or other
acggacatactcannnnng

SEQ ID NO: 24 - DNA, 21 bp, Homo sapiens
  modified_base (14)..(18): a, c, t, g, unknown or other
acggacatactcannnnngtt

SEQ ID NO: 25 - DNA, 21 bp, Homo sapiens
  modified_base (12)..(21): a, c, t, g, unknown or other
acggacatactnnnnnnnnnn

SEQ ID NO: 26 - DNA, 20 bp, Homo sapiens
acggacatactcatccatgt

SEQ ID NO: 27 - DNA, 18 bp, Homo sapiens
atggatgactatgtccgt

SEQ ID NO: 28 - DNA, 12 bp, Homo sapiens
acggacatagtc

SEQ ID NO: 29 - DNA, 21 bp, Homo sapiens
  modified_base (16)..(18): a, c, t, g, unknown or other
acggacatactcatcnnngtt

SEQ ID NO: 30 - DNA, 21 bp, Homo sapiens
  modified_base (12)..(20): a, c, t, g, unknown or other
acggacatactnnnnnnnnnt

SEQ ID NO: 31 - DNA, 18 bp, Homo sapiens
atggatgagtatgtccgt

SEQ ID NO: 32 - DNA, 19 bp, Homo sapiens
ctcgtcaacgaatcaaatt

SEQ ID NO: 33 - DNA, 19 bp, Homo sapiens
actccaagatgccaagtcg

SEQ ID NO: 34 - DNA, 19 bp, Homo sapiens
acggccgccccccggactc

SEQ ID NO: 35 - DNA, 21 bp, Homo sapiens
ggtaccacatgacaaaaccca

SEQ ID NO: 36 - DNA, 21 bp, Homo sapiens
ggtaccacatggcaaaaccca

SEQ ID NO: 37 - DNA, 28 bp, Homo sapiens
ggtgatccacgcaggtggttcgcagaag

SEQ ID NO: 38 - DNA, 19 bp, Homo sapiens
cgcaggtggttcgcagaag

SEQ ID NO: 39 - DNA, 25 bp, Homo sapiens
gatccacgcaggtggttcgcagaag

SEQ ID NO: 40 - DNA, 14 bp, Homo sapiens
gtggttcgcagaag

SEQ ID NO: 41 - DNA, 11 bp, Homo sapiens
gtggttcgcag

SEQ ID NO: 42 - DNA, 21 bp, Homo sapiens
ggtgatccacgcaggtggttc

SEQ ID NO: 43 - DNA, 19 bp, Homo sapiens
gctgccgtccgtatgagat

SEQ ID NO: 44 - DNA, 19 bp, Homo sapiens
acaggtgctttcggttgca

SEQ ID NO: 45 - DNA, 19 bp, Homo sapiens
acaggtgctctcggttgca

SEQ ID NO: 46 - DNA, 19 bp, Homo sapiens
ggtgatccacgcaggtggt

SEQ ID NO: 47 - DNA, 27 bp, Homo sapiens
ggtgatccacgcaggtggttccagaag

SEQ ID NO: 48 - DNA, 24 bp, Homo sapiens
ggtgatccacgcaggtggttcgca